(19) United States
(12) Patent Application Publication
(10) Pub. No.: US 2026/0076573 A1
(43) Pub. Date: Mar. 19, 2026

(54) BIOLOGICAL INFORMATION DETECTION DEVICE

(71) Applicant: SEIKO EPSON CORPORATION, Tokyo (JP)

(72) Inventors: Hiroki KOBAYASHI, NAGANO-KEN (JP); Ayae SAWADO, KAI-SHI (JP); Tsukasa EGUCHI, MATSUMOTO-SHI (JP)

(73) Assignee: SEIKO EPSON CORPORATION, Tokyo (JP)

(21) Appl. No.: 19/329,603

(22) Filed: Sep. 16, 2025

(30) Foreign Application Priority Data
Sep. 18, 2024 (JP) 2024-160857

(51) Int. Cl. A61B 5/00

(52) U.S. Cl. CPC A61B 5/0059

(57) ABSTRACT

A biological information detection device includes a housing having a planar housing surface, a detection unit having a light receiving portion, a light-transmissive member having a planar portion, and a pressing force suppression unit disposed on the housing surface to surround the planar portion, and suppressing a pressing force applied to a subject by the planar portion. The pressing force suppression unit is formed using a material harder than the subject. A height of the pressing force suppression unit from the housing surface is higher than a height of the planar portion with reference to the housing surface. The pressing force suppression unit has a first inclined section in which the height of the pressing force suppression unit increases continuously or in a stepwise manner from an inner periphery side toward an outer periphery side.

The present application is based on, and claims priority from JP Application Serial Number 2024-160857, filed Sep. 18, 2024, the disclosure of which is hereby incorporated by reference herein in its entirety.

### BACKGROUND

### 1. Technical Field

The present disclosure relates to a biological information detection device.

### 2. Related Art

In related art, a biological information acquisition device that receives scattered light from a biological tissue as a measuring object and acquires biological information including a blood flow volume, a blood volume, a blood flow velocity, and a pulse rate in the biological tissue by is known (for example, see JP-A-2004-188224).

A pulse wave information measurement device described in JP-A-2004-188224 includes a device main body having a wristwatch structure, a sensor unit, and a cable that electrically couples the device main body and the sensor unit.

The device main body includes a display unit that displays pulse wave information such as a pulse rate, and is detachably attached to an arm of a subject by a wristband.

The sensor unit is detachably attached to a finger of the subject by a sensor fixing band.

The sensor unit includes a sensor frame, and a light emitting element, a light receiving element, and a light-transmissive plate disposed in the sensor frame. The light-transmissive plate is disposed on the front surface side of the sensor frame, that is, on the finger side of the sensor frame, and the light emitting element and the light receiving element are disposed inside the sensor frame and on the back surface side of the light-transmissive plate. When the sensor unit is attached to the finger of the subject, the sensor frame and the light-transmissive plate are pressed against the finger.

At the measurement, light is emitted from the light emitting element, the light is transmitted through the light-transmissive plate and is applied to the surface of the living body, and the scattered light reflected by the living tissue is transmitted through the light-transmissive plate and is received by the light receiving element. Then, the pulse wave information is obtained based on the information from the light receiving element.

In this pulse wave information measurement device, as shown in FIG. 20(a) of JP-A-2004-188224, the outer surface of the sensor frame of the sensor unit, which is the surface on the side in contact with the finger of the subject, is a flat surface.

Further, the outer surface of the light-transmissive plate, which is the surface on the side in contact with the finger of the subject, protrudes to the finger side from the outer surface of the sensor frame. That is, the outer surface of the sensor frame is recessed from the outer surface of the translucent plate to the side opposite to the finger.

As shown in FIG. 20(b), JP-A-2004-188224 also describes a structure in which the outer surface of the light-transmissive plate is recessed from the outer surface of the sensor frame to the side opposite to the finger, that is, a structure in which the outer surface of the sensor frame protrudes to the finger side from the outer surface of the light-transmissive plate.

JP-A-2004-188224 is an example of the related art.

In the pulse wave information measurement device described in JP-A-2004-188224, the outer surface of the sensor frame is the flat surface and is recessed or protrudes from the outer surface of the light-transmissive plate, and thus the detection accuracy is lower.

That is, when the outer surface of the sensor frame is the flat surface and is recessed from the outer surface of the light-transmissive plate, a large pressing force acts on the finger from the outer surface of the light-transmissive plate, whereby the blood vessel is crushed, the detection of the pulse wave signal becomes difficult, the sensitivity becomes lower, and the detection accuracy becomes lower. Further, the pressing force from the finger is concentrated on the light-transmissive plate, whereby the pressing force applied to the finger is changed even by a subtle body motion, and the detection accuracy becomes lower.

When the outer surface of the sensor frame is the flat surface and protrudes from the outer surface of the light-transmissive plate, an air layer intervenes between the finger and the light-transmissive plate, and thus the detection of the pulse wave signal becomes difficult, the sensitivity becomes lower, and the detection accuracy becomes lower.

### SUMMARY

A biological information detection device according to an aspect of the present disclosure includes a housing having a planar housing surface and configured to come into contact with a subject when biological information of the subject is measured, a detection unit disposed in the housing and having a light receiving portion configured to receive light from the subject, a light-transmissive member disposed at a side of the housing surface of the housing, and having a planar portion that is configured to transmit the light from the subject and come into contact with the subject when the biological information of the subject is measured, and a pressing force suppression unit disposed on the housing surface to surround the planar portion as seen from a Z-axis direction, when two axes parallel to the housing surface and orthogonal to each other are an X-axis and a Y-axis, and an axis orthogonal to the X-axis and the Y-axis is the Z-axis, configured to come into contact with the subject when the biological information is measured, and configured to suppress a pressing force applied to the subject by the planar portion, wherein the pressing force suppression unit is formed using a material harder than the subject, a height of a side of the pressing force suppression unit that comes into contact with the subject from the housing surface in the Z-axis direction is higher than a height of the planar portion with reference to the housing surface, and the pressing force suppression unit has a first inclined section in which the height of the pressing force suppression unit increases continuously or in a stepwise manner from an inner periphery side toward an outer periphery side of the pressing force suppression unit.

### BRIEF DESCRIPTION OF THE DRAWINGS

FIG. 1 is a perspective view showing a first embodiment of a biological information detection device of the present disclosure.

FIG. 2 is a block diagram of the biological information detection device shown in FIG. 1.

FIG. 3 is a bottom view of the biological information detection device shown in FIG. 1.

FIG. 4 is a cross-sectional view of the biological information detection device shown in FIG. 1.

FIG. 5 is a cross-sectional view taken along line A-A in FIG. 3, showing a pressing force suppression unit and a light-transmissive plate of the biological information detection device in FIG. 1.

FIG. 6 is a bottom view showing a second embodiment of the biological information detection device of the present disclosure.

FIG. 7 is a bottom view showing a third embodiment of the biological information detection device of the present disclosure.

FIG. 8 is a bottom view showing a fourth embodiment of the biological information detection device of the present disclosure.

FIG. 9 is a cross-sectional view showing a pressing force suppression unit in a fifth embodiment of the biological information detection device of the present disclosure, and illustrates a portion corresponding to a unit pressing force suppression portion of the pressing force suppression unit in the first embodiment shown in FIG. 5 on the right side in FIG. 5.

FIG. 10 is a cross-sectional view showing a pressing force suppression unit in a sixth embodiment of the biological information detection device of the present disclosure, and illustrates a portion corresponding to the unit pressing force suppression unit of the pressing force suppression unit in the first embodiment shown in FIG. 5 on the right side in FIG. 5.

FIG. 11 is a cross-sectional view showing a pressing force suppression unit in a seventh embodiment of the biological information detection device of the present disclosure, and illustrates a portion corresponding to the unit pressing force suppression unit of the pressing force suppression unit in the first embodiment shown in FIG. 5 on the right side in FIG. 5.

FIG. 12 is a cross-sectional view showing a pressing force suppression unit in an eighth embodiment of the biological information detection device of the present disclosure, and illustrates a portion corresponding to the unit pressing force suppression unit of the pressing force suppression unit in the first embodiment shown in FIG. 5 on the right side in FIG. 5.

FIG. 13 is a cross-sectional view showing a pressing force suppression unit in a ninth embodiment of the biological information detection device of the present disclosure, and illustrates a portion corresponding to the unit pressing force suppression unit of the pressing force suppression unit in the first embodiment shown in FIG. 5 on the right side in FIG. 5.

FIG. 14 is a cross-sectional view showing a pressing force suppression unit in a tenth embodiment of the biological information detection device of the present disclosure, and illustrates a portion corresponding to the unit pressing force suppression unit of the pressing force suppression unit in the first embodiment shown in FIG. 5 on the right side in FIG. 5.

### DESCRIPTION OF EMBODIMENTS

Hereinafter, a biological information detection device of the present disclosure will be described in detail based on embodiments shown in the accompanying drawings.

### First Embodiment

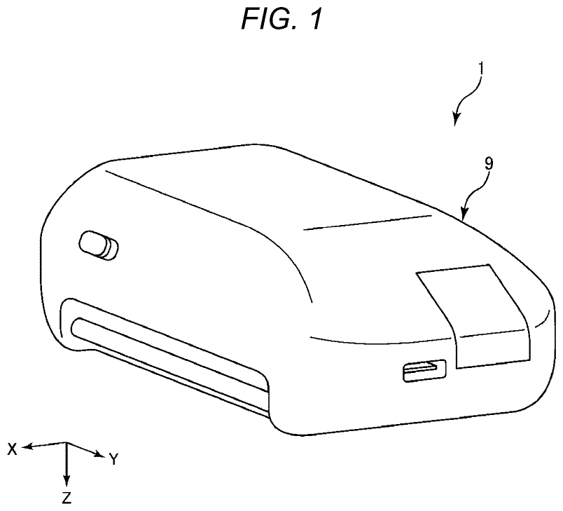

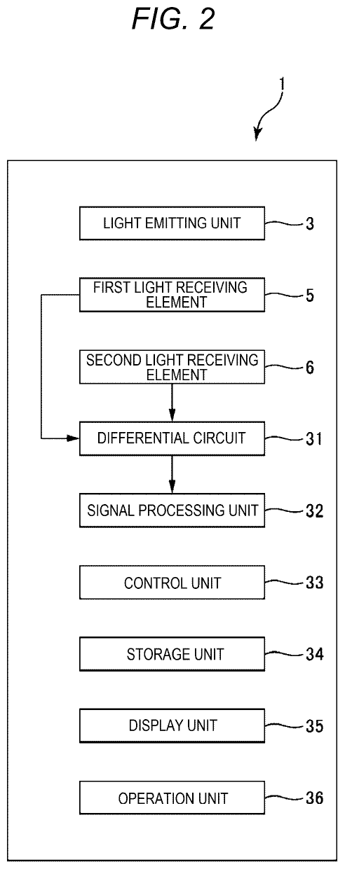

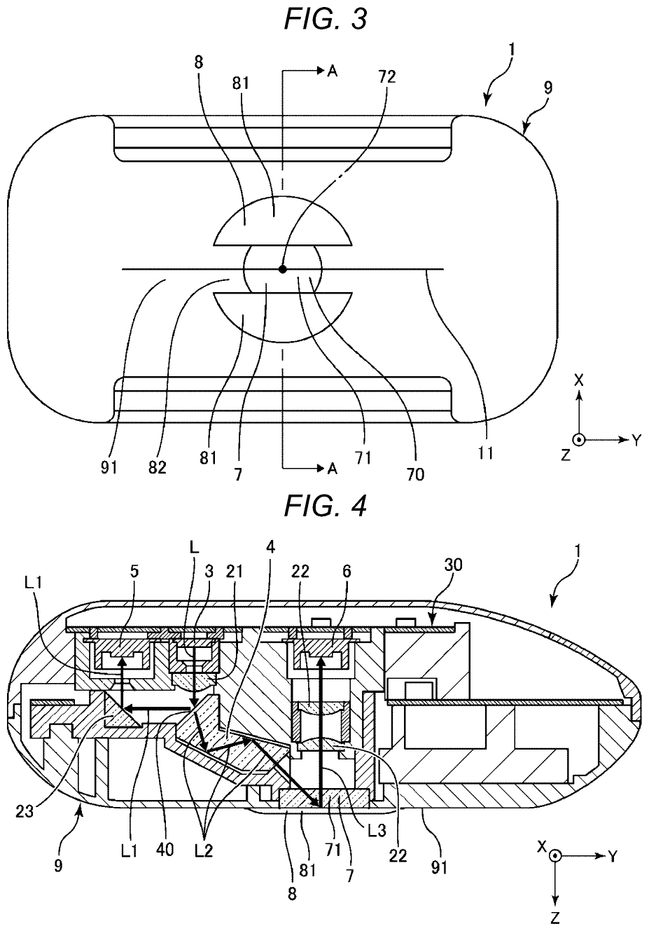

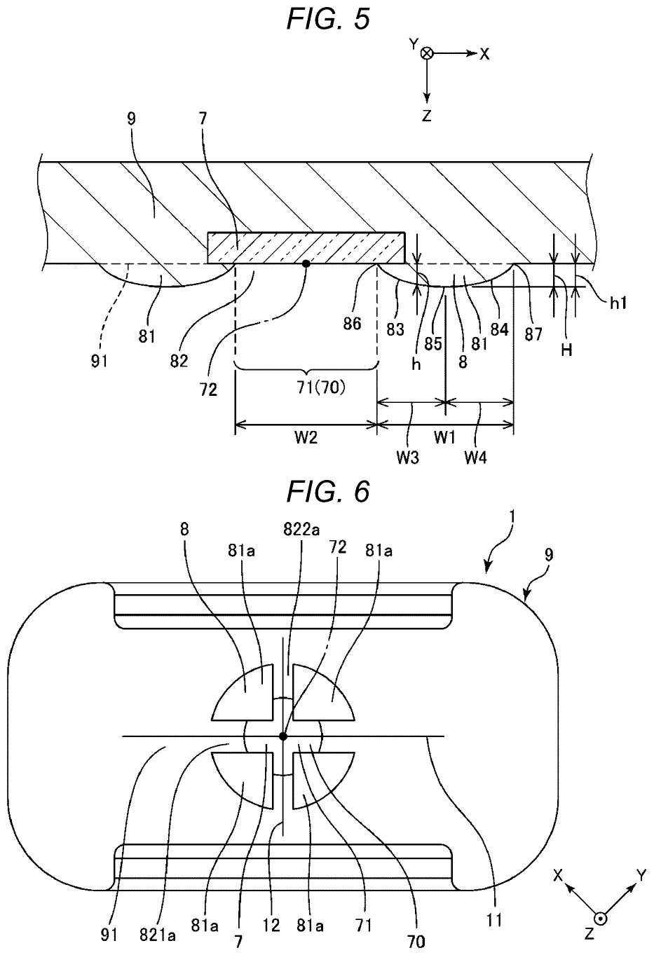

FIG. 1 is a perspective view showing a first embodiment of the biological information detection device of the present disclosure. FIG. 2 is a block diagram of the biological information detection device shown in FIG. 1. FIG. 3 is a bottom view of the biological information detection device shown in FIG. 1. FIG. 4 is a cross-sectional view of the biological information detection device shown in FIG. 1. FIG. 5 is a cross-sectional view taken along line A-A in FIG. 3, showing a pressing force suppression unit and a light-transmissive plate of the biological information detection device shown in FIG. 1.

In the specification, for convenience of description, the upsides in FIGS. 4 and 5 are referred to as “upper” or “upward” and the downsides are referred to as “lower” or “downward”.

As shown in the respective drawings except FIG. 2, as three axes orthogonal to one another, an X-axis, a Y-axis, and a Z-axis are shown. The pointer side of an arrow indicating each axis is “+: positive”, and the tail side is “−: negative”. Further, a direction parallel to the X-axis is also referred to as “X direction: X-axis direction”, a direction parallel to the Y-axis is also referred to as “Y direction: Y-axis direction”, and a direction parallel to the Z-axis is also referred to as “Z direction: Z-axis direction”.

The width direction of a biological information detection device 1, that is, the lateral direction of a housing 9 of the biological information detection device 1 is defined as the X direction. The length direction of the biological information detection device 1, that is, the longitudinal direction of the housing 9 of the biological information detection device 1 is defined as the Y direction. The thickness direction of the biological information detection device 1, that is, the height direction of the housing 9 of the biological information detection device 1 is defined as the Z direction.

The terms “planar” and “planar surface” as used in the specification of the present application are not limited to a case where the surface is completely flat, but include a case where the surface is slightly curved in a convex shape and a case where the surface is curved in a concave shape.

The biological information detection device 1 illustrated in FIG. 1 is a device that detects biological information of a subject as a measuring object that is a living body, that is, a device that measures and acquires biological information of the subject.

The biological information is information on the fluid of the subject, that is, biological fluid information, and examples thereof include a blood flow volume, a blood volume, a blood flow velocity, a pulse rate, a blood pressure, a pulse velocity, wave a degree of arteriosclerosis, and a volume pulse wave in the subject.

The form of the biological information detection device 1 is not particularly limited, but includes various forms such as a form attached to a subject for use and a stationary form. In the embodiment, application to a form attached to the subject for use is described. Specifically, application of the biological information detection device 1 to a form attached to an arm of a subject for use will be described. The part where the biological information detection device 1 is attached is not limited to the arm of the subject, but includes a finger, a wrist, and the like of the subject. When the device is attached to the arm, the wearing portion includes a forearm and an upper arm.

As illustrated in FIGS. 1, 3, and 4, the biological information detection device 1 has the housing 9 that houses and supports the respective units forming the biological information detection device 1. The housing 9 has a box shape, and has a rectangular parallelepiped outer shape with rounded corners. The housing 9 has a planar housing surface 91 coming into contact with the arm When the biological information of the subject is measured, that is, when the biological information detection device 1 is attached to the arm of the subject. That is, the lower surface of the housing 9 in FIG. 4 forms the planar housing surface 91. The housing surface 91 is parallel to the X-axis and the Y-axis, that is, parallel to the X-Y plane. The shape of the housing 9 is not limited thereto as long as the housing 9 has the planar housing surface 91.

Further, the biological information detection device 1 includes a circuit board 30 having various electronic components, circuits, and the like. The circuit board 30 is disposed, that is, housed in the housing 9.

As illustrated in FIGS. 2 and 4, the biological information detection device 1 includes a light emitting unit 3 that emits a laser beam L as measurement light applied to the subject, a first light receiving element 5, a second light receiving element 6, which is a light receiving unit that receives the light from the subject, a differential circuit 31, a signal processing unit 32 that generates biological information, a control unit 33 that controls driving of the biological information detection device 1, a storage unit 34 that stores various kinds of information, various programs, and the like, a display unit 35 that displays various kinds of information, and an operation unit 36, which is an input unit that gives various instructions and makes various kinds of input. The first light receiving element 5 and the second light receiving element 6 form a detection unit. The light emitting unit 3, the first light receiving element 5, the second light receiving element 6, the differential circuit 31, the signal processing unit 32, the control unit 33, the storage unit 34, the display unit 35, and the operation unit 36 are respectively electrically coupled to the circuit board 30, and predetermined members thereof are disposed on the circuit board 30.

The present disclosure is not limited to the above-described configuration, but may be a configuration in which a predetermined member among the units provided in the biological information detection device 1, for example, the display unit 35, the operation unit 36, and the like are disposed in a main body unit or the like disposed at positions apart from the housing 9.

The biological information detection device 1 includes, as an optical system, a prism 4 as a light branching member that branches light, a collimator lens 21, a condenser lens 22, a light-transmissive plate 7 as a light-transmissive member that transmits light, and a light reflection member 23. The prism 4, the collimator lens 21, the condenser lens 22, and the light reflection member 23 are disposed, that is, housed in the housing 9. The light-transmissive plate 7 is disposed in a lower portion of the housing 9, that is, in an end portion of the housing 9 at the + side in the Z direction, is supported by the housing 9, and is partially exposed to the outside as illustrated in FIGS. 3 and 5.

The circuit board 30 is disposed in an upper portion of the housing 9, that is, in an end portion of the housing 9 at the − side in the Z direction, and the light emitting unit 3, the first light receiving element 5, and the second light receiving element 6 are disposed on the circuit board 30. In this case, the first light receiving element 5 is disposed at the left side of the light emitting unit 3 in FIG. 4, that is, at the − side in the Y direction, and the second light receiving element 6 is disposed at the right side of the light emitting unit 3 in FIG. 4, that is, at the + side in the Y direction.

A polarization separation layer 40 is disposed on the incident surface of the laser beam L of the prism 4. The prism 4 is disposed in an optical path between the light emitting unit 3 and the light-transmissive plate 7. The prism 4 has a function of branching the laser beam L emitted from the light emitting unit 3 into a first luminous flux L1 and a second luminous flux L2. For example, the first luminous flux L1 is S-polarized light, and the second luminous flux L2 is P-polarized light. Instead of the prism 4, for example, a half mirror or the like may be provided.

As illustrated in FIGS. 3, 4, and 5, the light-transmissive plate 7 has a disk shape and is disposed at the + side in the Z direction of the second light receiving element 6 and at the housing surface 91 side of the housing 9, that is, an end portion at the + side in the Z direction of the housing 9. The outer peripheral portion of the light-transmissive plate 7 is covered with the housing 9 and a pressing force suppression unit 8 described later. The portion of the light-transmissive plate 7 exposed to the outside is a portion that comes into contact with the arm when the biological information of the subject is measured, that is, when the biological information detection device 1 is attached to the arm of the subject. The light-transmissive plate 7 has a function of pressing the arm with a predetermined force and a function of protecting the inside of the biological information detection device 1. Examples of the constituent material of the light-transmissive plate 7 include various glass materials and various resin materials.

As shown in FIG. 5, the light-transmissive plate 7 has a plate shape and has light transmissivity. The entire lower surface of the light-transmissive plate 7 in FIG. 5 is a planar surface. The second luminous flux L2 emitted from the prism 4 is transmitted through the light-transmissive plate 7, and scattered light L3 obtained from the subject is transmitted through the light-transmissive plate 7. Further, as shown in FIG. 3, although the shape of the light-transmissive plate 7 in a plan view, that is, the shape when viewed from the Z-axis direction is a circular shape, the shape is not limited thereto, but may be, for example, a polygonal shape such as a quadrangular shape, an elliptical shape, or the like.

In the present embodiment, the lower surface of the light-transmissive plate 7 in FIG. 5 is parallel to the X-axis and the Y-axis, that is, parallel to the X-Y plane. Therefore, in the present embodiment, the lower surface of the light-transmissive plate 7 in FIG. 5 is parallel to the housing surface 91 of the housing 9. However, the lower surface of the light-transmissive plate 7 in FIG. 5 may be slightly inclined by, for example, less than ±5° with respect to the housing surface 91.

In the present embodiment, the position of the lower surface of the light-transmissive plate 7 in FIG. 5 in the Z-axis direction coincides with the position of the housing surface 91 of the housing 9 in the Z-axis direction. However, the position of the lower surface of the light-transmissive plate 7 in FIG. 5 in the Z-axis direction may not coincide with the position of the housing surface 91 in the Z-axis direction.

In the present embodiment, the entire lower surface of the light-transmissive plate 7 in FIG. 5 is the planar surface, but the present disclosure is not limited thereto, as long as at least the entire lower part of an effective region 70 in FIG. 5 may be a planar surface.

In the light-transmissive plate 7, a portion having a surface exposed to the outside in the light-transmissive plate 7 is the effective region 70 that effectively functions, and is an effectively functioning planar portion 71. Hereinafter, the effectively functioning planar portion 71 is also simply referred to as “planar portion 71”.

As illustrated in FIGS. 3 and 5, the biological information detection device 1 includes the pressing force suppression unit 8 disposed on the housing surface 91.

The pressing force suppression unit 8 is disposed around the planar portion 71 of the light-transmissive plate 7. That is, when viewed from the Z-axis direction, the pressing force suppression unit 8 is disposed so as to surround the planar portion 71.

The pressing force suppression unit 8 has a function of coming into contact with the arm and suppressing the pressing force applied to the arm of the subject by the planar portion 71 when the biological information of the subject is measured, that is, when the biological information detection device 1 is attached to the arm of the subject. The pressing force suppression unit 8 will be described in detail later.

The collimator lens 21 is disposed in an optical path between the light emitting unit 3 and the incident surface of the laser beam L of the prism 4.

The condenser lens 22 is disposed in an optical path between the second light receiving element 6 and the light-transmissive plate 7.

The light reflection member 23 is disposed in an optical path between the first light receiving element 5 and the incident surface of the laser beam L of the prism 4. The light reflection member 23 has a function of reflecting the first luminous flux L1 reflected by the incident surface of the prism 4 toward the first light receiving element 5. Examples of the light reflection member 23 include a prism and a reflection plate.

Further, the light emitting unit 3 has a function of emitting laser beam L as measurement light. The light emitting unit 3 is not particularly limited, but includes a semiconductor laser.

The first light receiving element 5 has a function of receiving the first luminous flux L1. The first light receiving element 5 is not particularly limited, but includes a photodiode and a phototransistor.

The second light receiving element 6 has a function of receiving the scattered light L3 obtained from the arm when the second luminous flux L2 is incident on the arm as a part to be examined of the subject. The second light receiving element 6 is not particularly limited, but includes a photodiode and a phototransistor.

The differential circuit 31 has a function of generating a light detection signal based on the output of the first light receiving element 5 and the second light receiving element 6. That is, the differential circuit 31 converts detection currents output from the first light receiving element 5 and the second light receiving element 6 into voltage signals, generates a signal corresponding to the difference between the voltage signals, and outputs the signal as the light detection signal.

The signal processing unit 32 includes, for example, an arithmetic circuit such as a CPU (central processing unit) and can be implemented as one or more processors, and reads and executes various programs and the like stored in the storage unit 34. The signal processing unit 32 generates biological information by processing the light detection signal. A known method can be applied to the method of obtaining the biological information based on the light detection signal, and the description thereof will be omitted.

The control unit 33 includes, for example, an arithmetic circuit such as a CPU (central processing unit) and can be implemented as one or more processors, and reads and executes various programs and the like stored in the storage unit 34. Accordingly, various kinds of processing including control of the operation of the biological information detection 1, various calculations, various determinations, and the like is performed.

The processors implementing the control unit 33, the signal processing unit 32, and the like may be provided separately or may be entirely or partially shared.

The storage unit 34 stores various programs executable by CPU and the like. The storage unit 34 can store various types of data input from the outside. The storage unit 34 includes a volatile memory such as a RAM (random access memory) and a nonvolatile memory such as a ROM (read only memory). The storage unit 34 is not limited to an undetachable type, but may include a detachable external storage device.

The display unit 35 is an example of a notification unit for notification of information and, for example, has a function of displaying various kinds of information including the biological information. The display unit 35 is not particularly limited, but includes a liquid crystal display device and an organic EL display device.

The operation unit 36 is not particularly limited, but includes an operation button, an operation switch, and an operation dial. For example, the subject can give various instructions and make various kinds of input to the biological information detection device 1 by operating the operation unit 36.

Instead of the display unit 35 and the operation unit 36, a display input unit having both functions of the display unit 35 and the operation unit 36 may be used together with the display unit 35 and the operation unit 36. For example, as the display input device, a touch panel can be used.

Next, a procedure of detecting biological information of the subject by the biological information detection device 1 and an operation of the biological information detection device 1 will be described with reference to FIGS. 2 and 4.

First, the subject detachably attaches the biological information detection device 1 to the arm as the part to be examined by an attachment member (not illustrated). In this case, the arm is positioned on the +side in the Z direction of the biological information detection device 1, and the Y direction of the biological information detection device 1, that is, the longitudinal direction of the biological information detection device 1 is aligned with the extension direction of the arm. Thus, the arm is brought into contact with and pressed against the light-transmissive plate 7 and the pressing force suppression unit 8.

When the biological information is measured, that is, when the biological information is detected, the laser beam L is emitted from the light emitting unit 3, and the laser beam L becomes parallel light by the collimator lens 21, is incident on the incident surface of the prism 4, and is branched into the first luminous flux L1 and the second luminous flux L2. That is, the laser beam L is branched into the first luminous flux L1 reflected by the incident surface of the prism 4 and the second luminous flux L2 transmitted through the incident surface.

The first luminous flux L1 is reflected by the light reflection member 23, is incident on the first light receiving element 5, and is received by the first light receiving element 5. A detection current corresponding to the amount of received light is output from the first light receiving element 5 to the differential circuit 31.

The second luminous flux L2 is reflected within the prism 4, emitted from the emission surface, transmitted through the light-transmissive plate 7, and incident on, that is, applied to the arm of the subject. As a result, the scattered light L3 is emitted from the arm of the subject. The scattered light L3 is condensed by the condenser lens 22, is incident on the second light receiving element 6, and is received by the second light receiving element 6. A detection current corresponding to the amount of received light is output from the second light receiving element 6 to the differential circuit 31.

The differential circuit 31 converts the detection currents output from the first light receiving element 5 and the second light receiving element 6 into voltage signals, generates a signal corresponding to the difference between the voltage signals, and outputs the signal as a light detection signal to the signal processing unit 32.

The signal processing unit 32 generates predetermined biological information by processing the light detection signal. Examples of the biological information include a blood flow volume, a blood volume, a blood flow velocity, a pulse rate, blood pressure, a pulse wave velocity, a degree of arteriosclerosis, and a volume pulse wave.

The obtained biological information is stored in the storage unit 34 and read out as necessary. The obtained biological information is displayed by the display unit 35. Thus, the subject can grasp the biological information.

Next, the pressing force suppression unit 8 will be described.

The pressing force suppression unit 8 is formed using a material harder than the arm of the subject as a measuring object. Examples of the constituent material of the pressing force suppression unit 8 include various resin materials, various metal materials, and various ceramics.

Further, as shown in FIGS. 3 and 5, the pressing force suppression unit 8 has two unit pressing force suppression portions 81, and the overall shape of the pressing force suppression unit 8 is a shape obtained by dividing a member having an annular shape or a disk shape into two portions, more precisely, a shape obtained by equally dividing the member into two portions.

Specifically, the unit pressing force suppression portions 81 have the same shape, and are disposed apart from each other. When viewed from the Z-axis direction, the respective unit pressing force suppression portions 81 are disposed so as to surround the planar portion 71. Further, when viewed from the Z-axis direction, the shape of the unit pressing force suppression portion 81 is a shape obtained by removing the diameter side of a semicircle in parallel to the diameter. Furthermore, when viewed from the Z-axis direction, the unit pressing force suppression portions 81 are symmetrically disposed. That is, when viewed from the Z-axis direction, the unit pressing force suppression portions 81 are disposed point-symmetrically with respect to a center 72 of the planar portion 71 of the light-transmissive plate 7. In other words, when viewed from the Z-axis direction, the unit pressing force suppression portions 81 are disposed line-symmetrically with respect to a straight line 11 passing through the center 72 and parallel to the Y-axis.

As a result, a slit 82 penetrating the pressing force suppression unit 8 in the Y-axis direction as a direction orthogonal to the Z-axis is formed between the two unit pressing force suppression portions 81. That is, the slit 82 extends in the Y-axis direction, and the Y-axis direction is the longitudinal direction of the slit 82. The width of the slit 82, that is, the length of the slit 82 in the X-axis direction is constant along the Y-axis.

The slit 82 is disposed such that the longitudinal direction of the slit 82 is aligned with the blood flow direction of the arm of the subject when the biological information of the subject is measured, that is, when the biological information detection device 1 is attached to the arm of the subject. Accordingly, an impairment of blood circulation in the arm of the subject can be prevented by the pressing force suppression unit 8, and the biological information can be accurately detected.

Since the configurations of the unit pressing force suppression portions 81 are all the same, one unit pressing force suppression portion 81, that is, the unit pressing force suppression portion 81 on the right side in FIG. 5 will be representatively described below with reference to FIG. 5.

FIG. 5 is a cross-sectional view taken along line A-A in FIG. 3, illustrating the pressing force suppression unit and the light-transmissive plate of the biological information detection device illustrated in FIG. 1. The cross section shown in FIG. 5 is a cross section in a plane passing through the center 72 of the planar portion 71 of the light-transmissive plate 7 when viewed from the Z-axis direction and parallel to the X-axis and the Z-axis.

In the pressing force suppression unit 8, in the cross section illustrated in FIG. 5, the surface of the unit pressing force suppression portion 81 on the side in contact with the arm of the subject, that is, the surface of the unit pressing force suppression portion 81 as the lower surface of the unit pressing force suppression portion 81 in FIG. 5 has a convex curved shape. Hereinafter, the surface of the unit pressing force suppression portion 81 on the side in contact with the arm of the subject and the lower surface of the unit pressing force suppression portion 81 in FIG. 5 are also referred to as “the surface of the unit pressing force suppression portion 81”.

Specifically, the unit pressing force suppression portion 81 includes a first inclined section 83 in which the height of the unit pressing force suppression portion 81 continuously increases toward the + side in the X direction, that is, from the inner periphery side toward the outer periphery side of the unit pressing force suppression portion 81, and a second inclined section 84 in which the height of the unit pressing force suppression portion 81 continuously decreases from the inner periphery side toward the outer periphery side of the unit pressing force suppression portion 81. In the cross section shown in FIG. 5, each of the surface of the first inclined section 83 and the surface of the second inclined section 84 has a convex curved shape. The height of the unit pressing force suppression portion 81 is also the height of the pressing force suppression unit 8.

Further, the second inclined section 84 is disposed on the + side in the X direction with respect to the first inclined section 83, that is, on the outer periphery side of the unit pressing force suppression portion 81 with respect to the first inclined section 83, and immediately behind the first inclined section 83. A section between the first inclined section 83 and the second inclined section 84 is a top section 85 where the height of the unit pressing force suppression portion 81 is the maximum.

By setting the shape of the unit pressing force suppression unit 81 as described above, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

The unit pressing force suppression portion 81 has a bilaterally symmetrical shape in FIG. 5 with respect to the position of the top section 85. That is, the first inclined section 83 and the second inclined section 84 of the unit pressing force suppression portion 81 are line-symmetric with respect to a straight line passing through the top section 85 and parallel to the Z-axis. Note that the present disclosure is not limited thereto, but for example, the unit pressing force suppression portion 81 may be bilaterally asymmetrical.

In the present embodiment, although the radius of curvature of the surface of the first inclined section 83 is constant from an innermost peripheral part 86 to the top section 85 of the unit pressing force suppression portion 81, the present disclosure is not limited thereto, but the first inclined section 83 may have a part having a different radius of curvature.

Further, in the present embodiment, although the radius of curvature of the surface of the second inclined section 84 is constant from an outermost peripheral part 87 of the unit pressing force suppression portion 81 to the top section 85, the present disclosure is not limited thereto, but the second inclined section 84 may have a part having a different radius of curvature.

Here, the height of the pressing force suppression unit 8, that is, the height of the unit pressing force suppression portion 81 is a height of the surface of the unit pressing force suppression portion 81 from the housing surface 91 in the Z-axis direction when the housing surface 91 is used as a reference surface, and is indicated by a sign “h” in FIG. 5. The + side in the Z direction is “positive direction of the height”.

The height of the planar portion 71 of the light-transmissive plate 7 is the height of the surface of the planar portion 71 from the housing surface 91 in the Z-axis direction when the housing surface 91 is used as a reference surface. In the present embodiment, the height of the surface of the planar portion 71 is 0.

In the pressing force suppression unit 8, a height h1 of the unit pressing force suppression portion 81 as the height of the pressing force suppression unit 8 is higher than a height h2 of the planar portion 71. In this case, the height h1 is the maximum height of the surface of the unit pressing force suppression portion 81, that is, the height of the top section 85. In the present embodiment, the height h2 is 0, and the height h1 is larger than 0.

A height h3 of the innermost peripheral part 86 of the unit pressing force suppression portion 81 is equal to the height h2 of the planar portion 71. In the present embodiment, the height h3 is 0.

A height h4 of the outermost peripheral part 87 of the unit pressing force suppression portion 81 is equal to the height h2 of the planar portion 71. In the present embodiment, the height h4 is 0.

Since each of the height h2, the height h3, and the height h4 is 0, the signs “h2”, “h3”, and “h4” are not shown in the drawing.

Since the above-described pressing force suppression unit 8 is provided, the pressing force suppression unit 8 formed using the material harder than the arm of the subject is pressed against the arm of the subject at the position surrounding the planar portion 71 and pressing against the arm of the subject is generated at the position surrounding the planar portion 71, however, the pressing against the arm of the subject in the planar portion 71 is restricted. That is, the pressing force against the arm of the subject in the planar portion 71 can be set to the diastolic blood pressure of the subject or less, for example, 50 mmHg or less. Accordingly, at the position corresponding to the planar portion 71, crushing of the blood vessel of the subject can be suppressed, and an impairment of blood circulation in the arm of the subject can be suppressed. Further, pressing against the arm of the subject in the pressing force suppression unit 8 can be accurately performed, and a change in the body motion of the subject and a change in the pressing force by the biological information detection device 1 can be suppressed.

Since the height h3 of the innermost peripheral part 86 of the unit pressing force suppression portion 81 is equal to the height h2 of the planar portion 71, the planar portion 71 can be accurately brought into contact with the arm of the subject, and the air entering and intervening between the planar portion 71 and the arm of the subject due to the body motion of the subject can be suppressed.

Accordingly, the biological information can be accurately detected.

In the cross section shown in FIG. 5, a distance between the inner periphery and the outer periphery of the pressing force suppression unit 8 in the X-axis direction, that is, a distance between the innermost peripheral part 86 and the outermost peripheral part 87 of the unit pressing force suppression portion 81 in the X-axis direction is referred to as W1. A length of the planar portion 71 of the light-transmissive plate 7 in the X-axis direction is referred to as W2. A difference between the height h1 of the unit pressing force suppression portion 81 as the height of the pressing force suppression unit 8 and the height h2 of the planar portion 71 is referred to as H.

It is preferable that relationships of W2>H and W1>H are satisfied. It is preferable that W2 is from 1.5 times to 10 times the spot diameter of the laser beam L.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

It is preferable that the following relationships are satisfied.

$$0.05 \cdot W 2 < H < 0.5 \cdot W 2$$

$$0.2 \cdot W 2 < W 1 < 3 \cdot W 2$$

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

When H is larger than “0.5·W2”, the planar portion 71 is less likely to come into contact with the arm of the subject, and the measurement of biological information becomes unstable depending on other conditions.

When H is smaller than “0.05·W2”, it is difficult to sufficiently suppress the pressing force of the planar portion 71 against of the subject depending on other conditions.

The relationships among W1, W2, H and the spot diameter of the laser beam L are not limited to the relationships described above in the present disclosure.

In the cross section illustrated in FIG. 5, a distance between the inner periphery of the pressing force suppression unit 8 and the top section 85 in the X-axis direction, that is, a distance between the innermost peripheral part 86 of the unit pressing force suppression portion 81 and the top section 85 in the X-axis direction is referred to as W3. A distance between the outer periphery of the pressing force suppression unit 8 and the top section 85 in the X-axis direction, that is, a distance between the outermost peripheral part 87 of the unit pressing force suppression portion 81 and the top section 85 in the X-axis direction is referred to as W4. A height of the top section 85 of the unit pressing force suppression portion 81 as the height of the top section 85 of the pressing force suppression portion 8 is referred to as h5.

Although h5/W3 is not particularly limited, but appropriately set according to various conditions, and is preferably from 0.15 to 0.375, and more preferably from 0.03 to 0.188.

Accordingly, pressing against the arm of the subject in the pressing force suppression unit 8 can be accurately performed, and the air entering and intervening between the planar portion 71 and the arm of the subject due to the body motion of the subject can be suppressed. Further, biological tissues of the arm of the subject can be easily collected in the planar portion 71.

Although h5/W4 is not particularly limited, but is appropriately set according to various conditions, and is preferably from 0.15 to 0.375, and more preferably from 0.03 to 0.188.

Accordingly, pressing against the arm of the subject in the pressing force suppression unit 8 can be accurately performed, and the air entering and intervening between the planar portion 71 and the arm of the subject due to the body motion of the subject can be suppressed.

Note that h5/W3 and h5/W4 are equal in the present embodiment, but may be different.

Although the radius of curvature of the surface of the first inclined section 83 is not particularly limited, but is appropriately set according to various conditions, and is preferably from 14.5 mm to 362.5 mm, and more preferably from 29.0 mm to 182.2 mm.

Accordingly, pressing against the arm of the subject in the pressing force suppression unit 8 can be accurately performed, and the air entering and intervening between the planar portion 71 and the arm of the subject due to the body motion of the subject can be suppressed. Further, biological tissues of the arm of the subject can be easily collected in the planar portion 71.

Although the radius of curvature of the surface of the second inclined section 84 is not particularly limited, but is appropriately set according to various conditions, and is preferably from 14.5 mm to 362.5 mm, and more preferably from 29.0 mm to 182.2 mm.

Accordingly, pressing against the arm of the subject in the pressing force suppression unit 8 can be accurately performed, and the air entering and intervening between the planar portion 71 and the arm of the subject due to the body motion of the subject can be suppressed.

The radius of curvature of the surface of the first inclined section 83 and the radius of curvature 4 of the surface of the second inclined section 84 are equal in the present embodiment, but may be different.

The pressing force suppression unit 8 is hereinabove described.

In the present embodiment, although the unit pressing force suppression portion 81 does not have a planar section parallel to the housing surface 91, the portion is not limited thereto, but may have a planar section parallel to the housing surface 91 in a part thereof.

In the present embodiment, although, in the first inclined section 83 of the unit pressing force suppression portion 81, the height h of the unit pressing force suppression portion 81 continuously increases from the inner periphery side toward the outer periphery side of the unit pressing force suppression portion 81, the section is not limited thereto, but may have a part in which the height increases in a stepwise manner, or may have a part in which the height continuously increases and a part in which the height increases in a stepwise manner.

In the present embodiment, although, in the second inclined section 84 of the unit pressing force suppression portion 81, the height h of the unit pressing force suppression portion 81 continuously decreases from the inner periphery side toward the outer periphery side of the unit pressing force suppression portion 81, the section is not limited thereto, but may have a part in which the height decreases in a stepwise manner, or may have a part in which the height continuously decreases and a part in which the height decreases in a stepwise manner.

Further, in the present embodiment, although the configurations of the unit pressing force suppression portions 81 are all the same, the configurations are not limited thereto, but part or all of the configurations may be different.

In the present embodiment, although, when viewed from the Z-axis direction, the unit pressing force suppression portions 81 are disposed point-symmetrically and line-symmetrically, the portions are not limited thereto, but may be disposed asymmetrically.

In the present embodiment, although the number of the unit pressing force suppression portions 81 of the pressing force suppression unit 8 is two, the number is not limited thereto as long as the number is two or more, but may be three or more, and other configuration examples will be described in other embodiments to be described later.

As described above, according to the biological information detection device 1, the biological information can be accurately detected.

In the present embodiment, although, in the cross section shown in FIG. 5, the surface of the second inclined section 84 has the convex curved shape, the present disclosure is not limited thereto, but for example, the surface of the second inclined section 84 may have a concave curved shape or a linear shape.

The light-transmissive plate 7, in particular, the planar portion 71 of the light-transmissive plate 7 may be made formed using a soft material such as rubber. In this case, the entire planar portion 71 may be formed using a soft material, or the central part of the planar portion 71 may be formed using a soft material and the surrounding part may be formed using a hard material. Accordingly, the pressing force of the planar portion 71 against the arm of the subject can be reduced.

### Second Embodiment

FIG. 6 is a bottom view of the biological information detection device according to the second embodiment. In the present embodiment, regarding the X-axis, the Y-axis, and the Z-axis in the drawing, the X-axis and the Y-axis are rotated by a predetermined angle around the Z-axis with respect to those of the first embodiment.

Hereinafter, the second embodiment will be described with a focus on the differences from the above-described embodiment and the description of the same matters will be omitted. Note that each configuration whose description is omitted can be applied to the second embodiment.

The second embodiment is the same as the first embodiment except that the configuration of the pressing force suppression unit 8 is different.

As illustrated in FIG. 6, in the biological information detection device 1 of the second embodiment, the pressing force suppression unit 8 includes four unit pressing force suppression portions 81a, and the overall shape of the pressing force suppression unit 8 is a shape obtained by dividing a member having an annular shape or a disk shape into four portions, more precisely, a shape obtained by equally dividing the member into four portions.

Specifically, the unit pressing force suppression portions 81a have the same shape, and are disposed apart from one another. When viewed from the Z-axis direction, the respective unit pressing force suppression portions 81a are disposed so as to surround the planar portion 71. When viewed from the Z-axis direction, the unit pressing force suppression portion 81a has a fan shape with a central angle of 90°. When viewed from the Z-axis direction, the unit pressing force suppression portions 81a are symmetrically disposed. That is, when viewed from the Z-axis direction, each unit pressing force suppression portion 81a is disposed point-symmetrically with the corresponding unit pressing force suppression portion 81a with respect to the center 72 of the planar portion 71 of the light-transmissive plate 7. In other words, when viewed from the z-axis direction, each unit pressing force suppression portion 81a is disposed line-symmetrically with the corresponding unit pressing force suppression portion 81a with respect to the straight line 11 passing through the center 72 and parallel to the longitudinal direction of the biological information detection device 1. When viewed from the Z-axis direction, each unit pressing force suppression portion 81a is disposed line-symmetrically with the corresponding unit pressing force suppression portion 81a with respect to the straight line 12 passing through the center 72 and parallel to the lateral direction of the biological information detection device 1.

Accordingly, a slit 821a penetrating the pressing force suppression unit 8 in the longitudinal direction of the biological information detection device 1 as a direction orthogonal to the Z-axis and a slit 822a penetrating the pressing force suppression unit 8 in the lateral direction of the biological information detection device 1 as a direction orthogonal to the Z-axis are formed between two adjacent unit pressing force suppression portions 81a. That is, the slit 821a extends in the longitudinal direction of the biological information detection device 1, and the longitudinal direction of the biological information detection device 1 is the longitudinal direction of the slit 821a. The width of the slit 821a, that is, the length of the slit 821a in the lateral direction is constant along the straight line 11. The slit 822a extends in the lateral direction of the biological information detection device 1, and the lateral direction of the biological information detection device 1 is the longitudinal direction of the slit 822a. The width of the slit 822a, that is, the length of the slit 822a in the lateral direction is constant along the straight line 12. Further, the slit 821a and the slit 822a communicate with each other in their respective central parts.

The biological information detection device 1 can be selected to be attached to the arm of the subject such that the longitudinal direction of the biological information detection device 1 is the extension direction of the arm, or to be attached to the arm of the subject such that the lateral direction of the biological information detection device 1 is the extension direction of the arm.

When the biological information detection device 1 is attached to the arm of the subject such that the longitudinal direction thereof is the extension direction of the arm, the longitudinal direction of the slit 821a is aligned with the blood flow direction of the arm of the subject.

When the biological information detection device 1 is attached to the arm of the subject such that the lateral direction thereof is the extension direction of the arm, the longitudinal direction of the slit 822a is aligned with the blood flow direction of the arm of the subject.

Accordingly, an impairment of blood circulation in the arm of the subject can be prevented by the pressing force suppression unit 8, and the biological information can be accurately detected.

According to the second embodiment described above, the same effects as those of the above-described embodiment may be exerted.

The second embodiment can also be applied to fifth to tenth embodiments described later.

### Third Embodiment

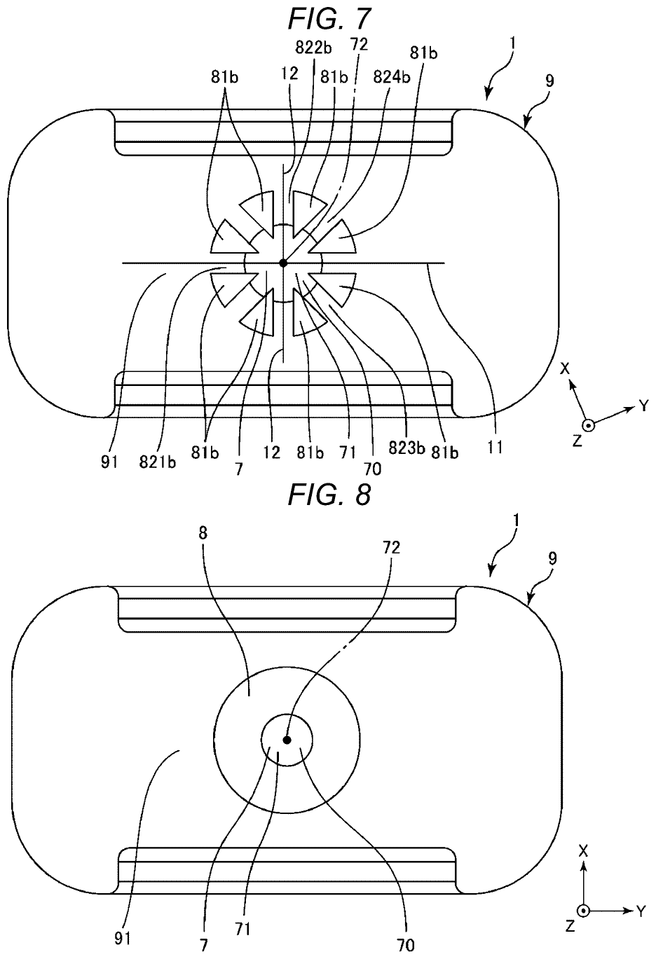

FIG. 7 is a bottom view of the biological information detection device according to a third embodiment. In the present embodiment, regarding the X-axis, the Y-axis, and the Z-axis in the drawing, the X-axis and the Y-axis are rotated by a predetermined angle around the Z-axis with respect to those of the first embodiment.

Hereinafter, the third embodiment will be described with a focus on the differences from the above-described embodiment and the description of the same matters will be omitted. Note that each configuration whose description is omitted can be applied to the third embodiment.

The third embodiment is the same as the first embodiment and the second embodiment except that the configuration of the pressing force suppression unit 8 is different.

As illustrated in FIG. 7, in the biological information detection device 1 of the third embodiment, the pressing force suppression unit 8 includes eight unit pressing force suppression portions 81b, and the overall shape of the pressing force suppression unit 8 is a shape obtained by dividing a member having an annular shape or a disk shape into eight portions, more precisely, a shape obtained by equally dividing the member into eight portions.

Specifically, the unit pressing force suppression portions 81b have the same shape, and are disposed apart from one another. When viewed from the Z-axis direction, the respective unit pressing force suppression portions 81b are disposed so as to surround the planar portion 71. When viewed from the Z-axis direction, the unit pressing force suppression portion 81b has a fan shape with a central angle of 45°. When viewed from the Z-axis direction, the unit pressing force suppression portions 81b are symmetrically disposed.

As a result, a slit 821b, a slit 822b, a slit 823b, and a slit 824b penetrating the pressing force suppression unit 8 in the direction orthogonal to the Z-axis are formed between two adjacent unit pressing force suppression units 81b. Further, the slit 821b, the slit 822b, the slit 823b, and the slit 824b communicate with one another in the central parts thereof.

According to the third embodiment described above, the same effects as those of the above-described embodiments may be exerted.

The third embodiment can also be applied to fifth to tenth embodiments described later.

### Fourth Embodiment

FIG. 8 is a bottom view of the biological information detection device according to a fourth embodiment.

Hereinafter, the fourth embodiment will be described with a focus on the differences from the above-described embodiment and the description of the same matters will be omitted. Note that each configuration whose description is omitted can be applied to the fourth embodiment.

The fourth embodiment is the same as the first embodiment, the second embodiment, and the third embodiment except that the configuration of the pressing force suppression unit 8 is different.

As illustrated in FIG. 8, in the biological information detection device 1 of the fourth embodiment, the pressing force suppression unit 8 has an annular shape.

When viewed from the Z-axis direction, the pressing force suppression unit 8 is disposed with the center thereof coinciding with the center 72 of the planar portion 71 of the light-transmissive plate 7 to surround the planar portion 71. When viewed from the Z-axis direction, the pressing force suppression unit 8 continuously surrounds the planar portion 71.

According to the fourth embodiment described above, the same effects as those of the above-described embodiments may be exerted.

The fourth embodiment can also be applied to fifth to tenth embodiments described later.

### Fifth Embodiment

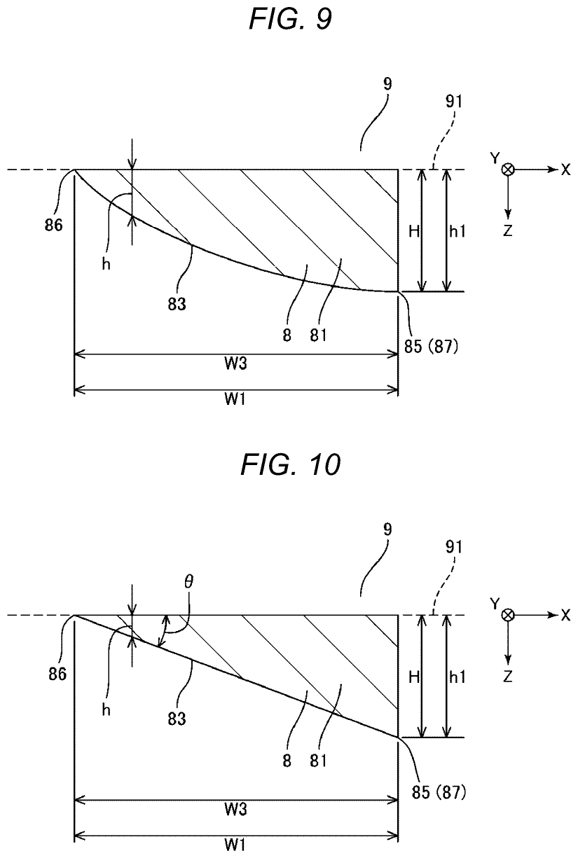

FIG. 9 is a cross-sectional view showing a pressing force suppression unit in a fifth embodiment of the biological information detection device of the present disclosure, and illustrates a portion corresponding to a unit pressing force suppression portion of the pressing force suppression unit in the first embodiment shown in FIG. 5 on the right side in FIG. 5.

Hereinafter, the fifth embodiment will be described with a focus on the differences from the above-described embodiment and the description of the same matters will be omitted. Note that each configuration whose description is omitted can be applied to the fifth embodiment.

The fifth embodiment is the same as the first embodiment except that the configuration of the pressing force suppression unit 8 is different.

As illustrated in FIG. 9, in the biological information detection device 1 of the fifth embodiment, the unit pressing force suppression portion 81 of the pressing force suppression unit 8 has the first inclined section 83, and the second inclined section 84 is omitted. The top section 85 of the unit pressing force suppression portion 81 and the outermost peripheral part 87 of the unit pressing force suppression portion 81 coincide with each other.

According to the fifth embodiment described above, the same effects as those of the above-described embodiments may be exerted.

### Sixth Embodiment

FIG. 10 is a cross-sectional view showing a pressing force suppression unit in a sixth embodiment of the biological information detection device of the present disclosure, and illustrates a portion corresponding to the unit pressing force suppression unit of the pressing force suppression unit in the first embodiment shown in FIG. 5 on the right side in FIG. 5.

Hereinafter, the sixth embodiment will be described with a focus on the differences from the above-described embodiment and the description of the same matters will be omitted. Note that each configuration whose description is omitted can be applied to the sixth embodiment.

The sixth embodiment is the same as the first embodiment except that the configuration of the pressing force suppression unit 8 is different.

As illustrated in FIG. 10, in the biological information detection device 1 of the sixth embodiment, the unit pressing force suppression portion 81 of the pressing force suppression unit 8 has the first inclined section 83, and the second inclined section 84 is omitted. The top section 85 of the unit pressing force suppression portion 81 and the outermost peripheral part 87 of the unit pressing force suppression portion 81 coincide with each other.

In the cross section shown in FIG. 10, the surface of the first inclined section 83 is linear. Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the cross section illustrated in FIG. 10, an angle formed by the surface of the first inclined section 83 and the housing surface 91 of the housing 9, that is, an inclination angle θ of the surface of the first inclined section 83 with respect to the housing surface 91 is not particularly limited, but is appropriately set according to various conditions, preferably from 1° to 5°.

Accordingly, pressing against the arm of the subject in the pressing force suppression unit 8 can be accurately performed, and the air entering and intervening between the planar portion 71 and the arm of the subject due to the body motion of the subject can be suppressed. Further, biological tissues of the arm of the subject can be easily collected in the planar portion 71.

In the present embodiment, the unit pressing force suppression portion 81 may include the second inclined section 84. In the cross section shown in FIG. 10, the surface of the second inclined section 84 has a linear shape similar to the surface of the first inclined section 83.

The unit pressing force suppression portion 81 has the second inclined section 84, and thus pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

According to the sixth embodiment described above, the same effects as those of the above-described embodiments may be exerted.

In the present embodiment, although, in the cross section shown in FIG. 10, the surface of the second inclined section 84 has the linear shape, the present disclosure is not limited thereto, but for example, the surface of the second inclined section 84 may have a concave curved shape.

### Seventh Embodiment

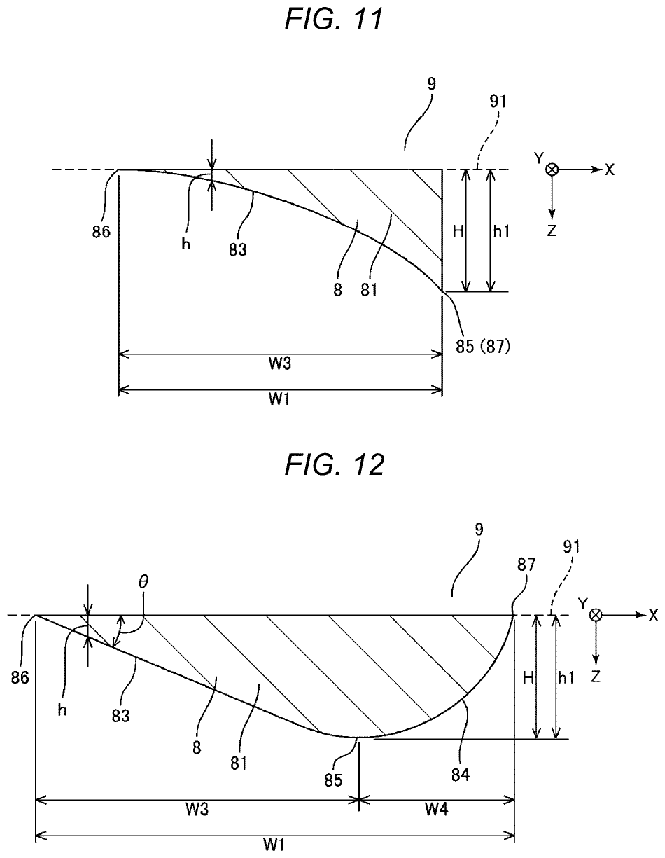

FIG. 11 is a cross-sectional view showing a pressing force suppression unit in a seventh embodiment of the biological information detection device of the present disclosure, and illustrates a portion corresponding to the unit pressing force suppression unit of the pressing force suppression unit in the first embodiment shown in FIG. 5 on the right side in FIG. 5.

Hereinafter, the seventh embodiment will be described with a focus on the differences from the above-described embodiment and the description of the same matters will be omitted. Note that each configuration whose description is omitted can be applied to the seventh embodiment.

The seventh embodiment is the same as the first embodiment except that the configuration of the pressing force suppression unit 8 is different.

As illustrated in FIG. 11, in the biological information detection device 1 of the seventh embodiment, the unit pressing force suppression portion 81 of the pressing force suppression unit 8 has the first inclined section 83, and the second inclined section 84 is omitted. The top section 85 of the unit pressing force suppression portion 81 and the outermost peripheral part 87 of the unit pressing force suppression portion 81 coincide with each other.

In the cross section shown in FIG. 11, the surface of the first inclined section 83 has a concave curved shape. Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the present embodiment, the unit pressing force suppression portion 81 may include the second inclined section 84. In the cross section shown in FIG. 11, the surface of the second inclined section 84 has a concave curved shape similar to the surface of the first inclined section 83.

The unit pressing force suppression portion 81 has the second inclined section 84, and thus pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

According to the seventh embodiment described above, the same effects as those of the above-described embodiments may be exerted.

In the present embodiment, although, in the cross section shown in FIG. 11, the surface of the second inclined section 84 has the concave curved shape, the present disclosure is not limited thereto, but for example, the surface of the second inclined section 84 may have a linear shape.

### Eighth Embodiment

FIG. 12 is a cross-sectional view showing a pressing force suppression unit in an eighth embodiment of the biological information detection device of the present disclosure, and illustrates a portion corresponding to the unit pressing force suppression unit of the pressing force suppression unit in the first embodiment shown in FIG. 5 on the right side in FIG. 5.

Hereinafter, the eighth embodiment will be described with a focus on the differences from the above-described embodiment and the description of the same matters will be omitted. Note that each configuration whose description is omitted can be applied to the eighth embodiment.

The eighth embodiment is the same as the first embodiment except that the configuration of the pressing force suppression unit 8 is different.

As illustrated in FIG. 12, in the biological information detection device 1 of the eighth embodiment, in the cross section illustrated in FIG. 12, the surface of the first inclined section 83 of the unit pressing force suppression portion 81 of the pressing force suppression unit 8 has a linear shape. Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the cross section illustrated in FIG. 12, the angle formed by the surface of the first inclined section 83 and the housing surface 91 of the housing 9, that is, the inclination angle θ of the surface of the first inclined section 83 with respect to the housing surface 91 is preferably from 1° to 5° as described in the sixth embodiment.

According to the eighth embodiment described above, the same effects as those of the above-described embodiments may be exerted.

### Ninth Embodiment

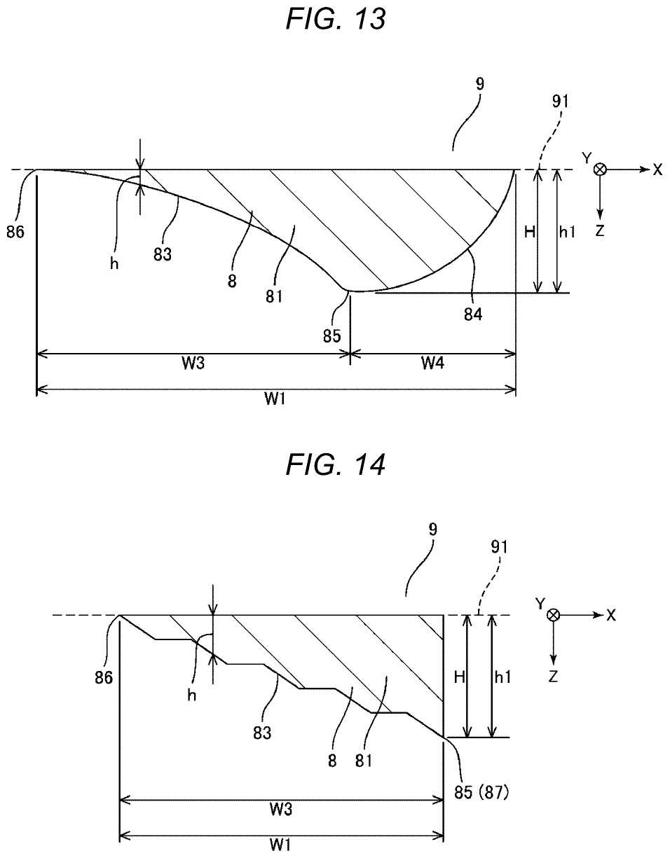

FIG. 13 is a cross-sectional view showing a pressing force suppression unit in a ninth embodiment of the biological information detection device of the present disclosure, and illustrates a portion corresponding to the unit pressing force suppression unit of the pressing force suppression unit in the first embodiment shown in FIG. 5 on the right side in FIG. 5.

Hereinafter, the ninth embodiment will be described with a focus on the differences from the above-described embodiment and the description of the same matters will be omitted. Note that each configuration whose description is omitted can be applied to the ninth embodiment.

The ninth embodiment is the same as the first embodiment except that the configuration of the pressing force suppression unit 8 is different.

As illustrated in FIG. 13, in the biological information detection device 1 of the ninth embodiment, in the cross section illustrated in FIG. 13, the surface of the first inclined section 83 of the unit pressing force suppression portion 81 of the pressing force suppression unit 8 has a concave curved shape. Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

According to the ninth embodiment described above, the same effects as those of the above-described embodiments may be exerted.

### Tenth Embodiment

FIG. 14 is a cross-sectional view showing a pressing force suppression unit in a tenth embodiment of the biological information detection device of the present disclosure, and illustrates a portion corresponding to the unit pressing force suppression unit of the pressing force suppression unit in the first embodiment shown in FIG. 5 on the right side in FIG. 5.

Hereinafter, the tenth embodiment will be described with a focus on the differences from the above-described embodiment and the description of the same matters will be omitted. Note that each configuration whose description is omitted can be applied to the tenth embodiment.

The tenth embodiment is the same as the first embodiment except that the configuration of the pressing force suppression unit 8 is different.

As illustrated in FIG. 14, in the biological information detection device 1 of the tenth embodiment, the unit pressing force suppression portion 81 of the pressing force suppression unit 8 has the first inclined section 83, and the second inclined section 84 is omitted. The top section 85 of the unit pressing force suppression portion 81 and the outermost peripheral part 87 of the unit pressing force suppression portion 81 coincide with each other.

In the first inclined section 83 of the unit pressing force suppression portion 81, the height h of the unit pressing force suppression portion 81 increases in a stepwise manner from the inner periphery side toward the outer periphery side of the unit pressing force suppression portion 81.

In the present embodiment, the unit pressing force suppression portion 81 may include the second inclined section 84. In the second inclined section 84, the height h of the unit pressing force suppression portion 81 decreases in a stepwise manner from the inner periphery side toward the outer periphery side of the unit pressing force suppression portion 81.

According to the tenth embodiment described above, the same effects as those of the above-described embodiments may be exerted.

In the present embodiment, when the unit pressing force suppression portion 81 includes the first inclined section 83 and the second inclined section 84, although the height h of the unit pressing force suppression portion 81 changes in a stepwise manner from the inner periphery side toward the outer periphery side of the unit pressing force suppression portion 81 in the first inclined section 83 and the second inclined section 84, the present disclosure is not limited thereto, but the height h may change in a stepwise manner in only one of the first inclined section 83 and the second inclined section 84.

As described above, the biological information detection device 1 includes the housing 9 coming into contact with the subject as the measuring object when biological information of the subject is measured and having the planar housing surface 91, the first light receiving element 5 and the second light emitting element 6 as a detection unit disposed in the housing 9 and having the light receiving portion that receives the diffused light L3 as the light from the subject, the light-transmissive plate 7 as a light-transmissive member disposed at a side of the housing surface 91 of the housing 9, and having the planar portion 71 that transmits the diffused light L3 as the light from the subject and comes into contact with the subject when the biological information of the subject is measured, and the pressing force suppression unit 8 disposed on the housing surface 91 to surround the planar portion 71 as seen from the Z-axis direction, when the two axes parallel to the housing surface and orthogonal to each other are the X-axis and the Y-axis, and the axis orthogonal to the X-axis and the Y-axis is the Z-axis, coming into contact with the subject when the biological information is measured, and suppressing a pressing force applied to the subject by the planar portion 71. The pressing force suppression unit 8 is formed using a material harder than the subject. Further, the height h1 of the side of the pressing force suppression unit 8 that comes into contact with the subject from the housing surface 91 in the Z-axis direction is higher than the height h2 of the planar portion 71 with reference to the housing surface 91. The pressing force suppression unit 8 has the first inclined section 83 in which the height h of the pressing force suppression unit 8 increases continuously or in a stepwise manner from the inner periphery side toward the outer periphery side of the pressing force suppression unit 8. The height h1 is the maximum height of the surface of the pressing force suppression unit 8, that is, the maximum height of the surface of the unit pressing force suppression portion 81.

According to the biological information detection device 1, the pressing force suppression unit 8 formed using the material harder than the arm of the subject is pressed against the arm of the subject at the position surrounding the planar portion 71 and pressing against the arm of the subject is generated at the position surrounding the planar portion 71, however, the pressing against the arm of the subject in the planar portion 71 is restricted. That is, the pressing force on the arm of the subject in the planar portion 71 can be set to be equal to or less than the diastolic blood pressure of the subject. Accordingly, at the position corresponding to the planar portion 71, crushing of the blood vessel of the subject can be suppressed, and an impairment of blood circulation in the arm of the subject can be suppressed. Further, pressing against the arm of the subject in the pressing force suppression unit 8 can be accurately performed, and a change in the body motion of the subject and a change in the pressing force by the biological information detection device 1 can be suppressed. Accordingly, the biological information can be accurately detected.

The biological information detection device 1 includes the light emitting unit 3 that is disposed in the housing 9 and emits the laser beam L as the measurement light transmitted through the light-transmissive plate 7 and applied to the subject.

In a cross section on a plane passing through the center 72 of the planar portion 71 as seen from the Z-axis direction and parallel to the X-axis and the Z-axis, when the distance between the inner periphery and the outer periphery of the pressing force suppression unit 8 in the X-axis direction is W1, the length of the planar portion 71 in the X-axis direction is W2, and the difference between the height h1 of the pressing force suppression unit 8 and the height h2 of the planar portion 71 is H, the relationships of W2>H and W1>H are satisfied, and W2 is from 1.5 times to 10 times the spot diameter of the laser beam L.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the biological information detection device 1, the pressing force suppression unit 8 includes the plurality of unit pressing force suppression portions 81 disposed to surround the planar portion 71 apart from each other when viewed from the Z-axis direction.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the biological information detection device 1, the pressing force suppression unit 8 includes the two unit pressing force suppression portions 81, and the slit 82 penetrating the pressing force suppression unit 8 in the Y-axis direction as the direction orthogonal to the Z-axis is formed between the two unit pressing force suppression portions 81. Further, the slit 82 is disposed such that the longitudinal direction of the slit 82 is aligned with the blood flow direction of the subject when the biological information is measured.

Accordingly, because of the slit 82, an impairment of blood circulation in the arm of the subject can be suppressed by the pressing force suppression unit 8, and the biological information can be accurately detected.

In the biological information detection device 1, the height h of the pressing force suppression unit 8 continuously increases in the first inclined section 83. The surface of the first inclined section 83 has the linear shape in the cross section on the plane passing through the center 72 of the planar portion 71 when viewed from the Z-axis direction and parallel to the X-axis and the Z-axis.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the biological information detection device 1, the height h of the pressing force suppression unit 8 continuously increases in the first inclined section 83. The surface of the first inclined section 83 has the convex curved shape in the cross section on the plane passing through the center 72 of the planar portion 71 when viewed from the Z-axis direction and parallel to the X-axis and the Z-axis.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the biological information detection device 1, the height h of the pressing force suppression unit 8 continuously increases in the first inclined section 83. The surface of the first inclined section has the concave curved shape in the cross section on the plane passing through the center 72 of the planar portion 71 when viewed from the Z-axis direction and parallel to the X-axis and the Z-axis.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the biological information detection device 1, the pressing force suppression unit 8 has the second inclined section 84 disposed closer to the outer periphery side of the pressing force suppression unit 8 than the first inclined section 83, in which the height h of the pressing force suppression unit 8 decreases continuously or in a stepwise manner from the inner periphery side toward the outer periphery side of the pressing force suppression unit 8.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the biological information detection device 1, the height h of the pressing force suppression unit 8 continuously decreases in the second inclined section 84. The surface of the second inclined section 84 has the linear shape in the cross section on the plane passing through the center 72 of the planar portion 71 when viewed from the Z-axis direction and parallel to the X-axis and the Z-axis.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

In the biological information detection device 1, the height h of the pressing force suppression unit 8 continuously decreases in the second inclined section 84. The surface of the second inclined section 84 has the convex curved shape in the cross section on the plane passing through the center 72 of the planar portion 71 when viewed from the Z-axis direction and parallel to the X-axis and the Z-axis.

Accordingly, pressing against the arm of the subject in the pressing force suppression unit 8 can be accurately performed, and the pressing force against the arm of the subject in the planar portion 71 can be easily set in an appropriate range, for example, equal to or less than the diastolic blood pressure of the subject.

As above, the biological information detection device of the present disclosure is described based on the illustrated embodiments, however, the present disclosure is not limited thereto. The configurations of the respective portions can be replaced by any configurations having the same functions. Further, any other configuration may be added.

Furthermore, the present disclosure can be a combination of any two or more configurations of the embodiments described above.

What is claimed is:

1. A biological information detection device, comprising:
a housing having a planar housing surface and configured to come into contact with a subject when biological information of the subject is measured;
a detection unit disposed in the housing and having a light receiving portion configured to receive light from the subject;
a light-transmissive member disposed at a side of the housing surface of the housing, and having a planar portion configured to transmit the light from the subject and configured to come into contact with the subject when the biological information of the subject is measured; and
a pressing force suppression unit disposed on the housing surface to surround the planar portion as seen from a Z-axis direction, when two axes parallel to the housing surface and orthogonal to each other are an X-axis and a Y-axis, and an axis orthogonal to the X-axis and the Y-axis is the Z-axis, configured to come into contact with the subject when the biological information is measured, and configured to suppress a pressing force applied to the subject by the planar portion, wherein
the pressing force suppression unit is formed using a material harder than the subject,
a height of a side of the pressing force suppression unit that comes into contact with the subject from the housing surface in the Z-axis direction is higher than a height of the planar portion with reference to the housing surface, and
the pressing force suppression unit has a first inclined section in which the height of the pressing force suppression unit increases continuously or in a stepwise manner from an inner periphery side toward an outer periphery side of the pressing force suppression unit.

2. The biological information detection device according to claim 1, further comprising: a light emitting unit disposed in the housing and configured to emit measurement light to be transmitted through the light-transmissive member and applied to the subject, wherein
when a distance between an inner periphery and an outer periphery of the pressing force suppression unit in the X-axis direction is W1, a length of the planar portion in the X-axis direction is W2, and a difference between the height of the pressing force suppression unit and the height of the planar portion is H in a cross section on a plane passing through a center of the planar portion when viewed from the Z-axis direction and parallel to the X-axis and the Z-axis, relationships of W2>H and W1>H are satisfied, and W2 is from 1.5 times to 10 times a spot diameter of the measurement light.

3. The biological information detection device according to claim 1, wherein
the pressing force suppression unit includes a plurality of unit pressing force suppression portions disposed to surround the planar portion apart from each other when viewed from the Z-axis direction.

4. The biological information detection device according to claim 3, wherein
the pressing force suppression unit includes two of the unit pressing force suppression portions,
a slit that penetrates the pressing force suppression unit in a direction orthogonal to the Z-axis is formed between the two unit pressing force suppression portions, and
the slit is disposed with a longitudinal direction of the slit aligned with a blood flow direction of the subject when the biological information is measured.

5. The biological information detection device according to claim 1, wherein
the height of the pressing force suppression unit continuously increases in the first inclined section, and
a surface of the first inclined section has a linear shape in a cross section on a plane passing through a center of the planar portion when viewed from the Z-axis direction and parallel to the X-axis and the Z-axis.

6. The biological information detection device according to claim 1, wherein
the height of the pressing force suppression portion continuously increases in the first inclined section, and
a surface of the first inclined section has a convex curved shape in a cross section on a plane passing through a center of the planar portion when viewed from the Z-axis direction and parallel to the X-axis and the Z-axis.

7. The biological information detection device according to claim 1, wherein
the height of the pressing force suppression portion continuously increases in the first inclined section, and
a surface of the first inclined section has a concave curved shape in a cross section on a plane passing through a center of the planar portion when viewed from the Z-axis direction and parallel to the X-axis and the Z-axis.

8. The biological information detection device according to claim 1, wherein
the pressing force suppression unit includes a second inclined section disposed closer to the outer periphery side of the pressing force suppression unit than the first inclined section, in which the height of the pressing force suppression unit decreases continuously or in a stepwise manner from the inner periphery side toward the outer periphery side of the pressing force suppression unit.

9. The biological information detection device according to claim 8, wherein
the height of the pressing force suppression unit continuously decreases in the second inclined section, and
a surface of the second inclined section has a linear shape in a cross section on a plane passing through a center of the planar portion when viewed from the Z-axis direction and parallel to the X-axis and the Z-axis.

10. The biological information detection device according to claim 8, wherein
the height of the pressing force suppression unit continuously decreases in the second inclined section, and
a surface of the second inclined section has a convex curved shape in a cross section on a plane passing through a center of the planar portion when viewed from the Z-axis direction and parallel to the X-axis and the Z-axis.
